(19) United States
(12) Patent Application Publication
(10) Pub. No.: US 2005/0260767 A1
(43) Pub. Date: Nov. 24, 2005

(54) Treatment with cytokines

(76) Inventors: Mario Clerici, Milan (IT); Giorgio Annoni, Milan (IT)

Correspondence Address: EDWARDS & ANGELL, LLP, P.O. BOX 55874, BOSTON, MA, 02205, US

(21) Appl. No.: 10/516,421

(22) PCT Filed: May 30, 2003
(86) PCT No.: PCT/GB03/02369

§ 371 Date: Jun. 30, 2005

(30) Foreign Application Priority Data
May 31, 2002 (GB) 0212648.0

(51) Int. Cl. G01N 33/543

(52) U.S. Cl. 436/518

(57) ABSTRACT

An inflammatory process is suggested to be involved in the pathogenesis of Alzheimer's disease (AD), a neurodegenerative disorder characterized by the presence of neuritic plaques within the cerebral cortex that are mainly composed of a small insoluble protein of 40-42 amino acids (amyloid protein). The biological correlates of this process are nevertheless not clear. Interleukin-10 (IL-10) is a cytokine that suppresses T lymphocytes and cell-mediated immunity in humans and mice and has potent anti-inflammatory properties. To verify if IL-10 production would be impaired in AD patients we stimulated PBMC of 47 patients and 25 age-matched healthy controls (HC) with a mitogen, a recall antigen or with amyloid peptides. IL-2 production was measured as well in the same cultural conditions. Results showed that amyloid-specific IL-10 generation is selectively and significantly reduced in AD patients (p=0.023). Analyses on the alleles of the IL-10 gene revealed that the genotype associated with high IL-10 production is extremely infrequent in AD individuals (2% vs. 28%). The presence of low/intermediate IL-10-producing genotypes (GCC/ATA; ATA/ATA) was associated with an earlier age at disease onset and (ACC/ACC; ACC/ATA) with an accelerated rate of disease progression. These data shed light on the biology of the inflammatory process involved in the pathogenesis of AD by showing that the presence of low-IL-10-allelic isoforms results in an amyloid-specific impairment of IL-10 production and is associated with the clinical severity of AD. These results lend support to the use of anti-inflammatory compounds in the therapy of this disease.

This invention relates to the use of cytokines in the diagnosis, treatment or prophylaxis of diseases. More particularly, the present invention relates to the use of cytokines to diagnose or treat non-neoplastic or non-leukaemic diseases such as autoimmune diseases or neurodegenerative disorders.

In the description which follows, the present invention will be described with particular reference to the most preferred embodiment of the invention which relates to the use of the cytokine interleukin-10 in the diagnosis, treatment or prophylaxis of the neurodegenerative disorder Alzheimer's disease. It is not intended to restrict the scope of the present invention to this one embodiment since the present invention finds equal utility in other disorders such as autoimmune diseases, for example multiple sclerosis, myasthenia gravis, systemic lupus erythematosus, diabetes mellitus and asthma, other neurodegenerative disorders for example Parkinson's disease, motor neurone disease and Alzheimer's disease; chronic inflammatory diseases such as rheumatoid arthritis; and other diseases where the modulation of T-Cell function is desirable such as HIV-infection and AIDS.

Similarly, the invention has utility with all cytokines, not solely interleukin-10 and hence it is intended to include cytokines such as interleukin-1 (α or β), interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-15, interleukin-16, interleukin-17, interferon-α, interferon-β, interferon-γ, TNF-α, TNF-β, G-CSF, GM-CSF, M-LSF, and TGF-β, in the scope of the present invention.

The major cause of cognitive decline in the elderly is Alzheimer's disease (AD). Because of longer life spans worldwide, the number of people that will be affected by AD is expected to triple over the next 50 years (1). AD is a clinical syndrome characterised by complex and heterogeneous pathogenic mechanisms. The recognised genetic factors include mutations of the gene encoding the amyloid precursor protein (2), presenilin 1 and 2 (3, 4), which account for a small part of familial and usually early-onset AD cases. Genetic factors have also been associated with the sporadic or non-familial form of the disease and the allele e4 of the apolipoprotein E (Apo E) significantly increases the risk of AD, but is neither necessary nor sufficient for the development of the disease (5-7). Therefore other genetic and environmental factors are likely to be implicated and are actively investigated.

Molecules that take part in the inflammatory cascade are of great interest, because inflammation has repeatedly been suggested to be associated with the neurodegenerative process characteristic of the AD brain (8). Thus, reactive astrocytosis is observed both in the cortex and in the hippocampus of these patients and the glial cells are also activated within or near the neuritic plaques. Over-expression of cytokines and other inflammatory molecules are common features of the AD brain pathology (9). Additionally, epidemiological studies showed that the long term use of non-steroid anti-inflammatory drugs is associated with a decreased incidence of AD in a co-twin control study (10) and several other clinical studies confirmed a decreased association of AD in individuals treated with anti-inflammatory drugs (11) including COX2 specific inhibitors (12). These findings support the hypothesis that inflammation might contribute to the neurodegeneration associated with AD (13).

In the attempt to better understand the biology of AD the possible role of several cytokines and chemokines has recently been investigated. Virtually all of the mediators analyzed in these studies, including IL-1b, IL-6, TNF-α, IL-8, TGF-β and macrophage inflammatory protein-1a (MIP-1a), have been suggested to be up-regulated in AD compared to non demented controls (14-18). On the contrary, conflicting results are obtained in relation to the immunomodulatory cytokine IL-10, a type-2 cytokine that suppresses T lymphocytes and cell-mediated immunity in humans and mice and has potent anti-inflammatory properties (19-21).

These studies considered each cytokine independently as gene polymorphisms and/or production, but never investigated the relationship between factors acting for and against inflammation, such as IL-10 and IL-6, in the same population sample.

It is worth recalling that single nucleotide polymorphisms (SNPs) in the promoter region of these two genes are known. The gene encoding IL-10, mapped to chromosome 1 between 1q31 and 1q32, is highly polymorphic. IL-10 production is correlated to biallelic polymorphisms at positions: −1082 (guanine to adenine substitution), −819 (thymine to cytosine substitution), and −592 (adenine to cytosine substitution). The polymorphism at position −1082 lies within an Ets (E-twenty-six specific)-like recognition site and may affect the binding of this transcriptional factor and therefore alter transcription activation; the −1082 A allele correlates with IL-10 generation after stimulation of T cells in vitro (57), while polymorphisms at positions −819 and −592 do not seem to be involved. The IL-6 gene in humans is organised in five exons and four introns and maps to the short arm of chromosome 7 (7p21) (50, 73). The involvement of IL-6 in many biological functions is paralleled by genetic associations of its allelic variants with several physiological and pathophysiological conditions. Two of its polymorphic sites have been frequently used for genetic association studies: a multiallelic variable number of tandem repeats (VNTR) polymorphism in the 3′ flanking region (AT repeats) and a biallelic G-to-C polymorphism of the promoter at the position −174. The G/C single nucleotide polymorphism (SNP) seems to be associated with varying blood levels and transcription rates of IL-6 (54, 56, 68).

In the light of these considerations and on the basis of a case-control association study in Italian sporadic late-onset AD patients and matched healthy controls (HC), the present inventors evaluated whether IL-10 and IL-6 SNPs were related with the development of AD. The results shed further light on the inflammatory pathogenic hypothesis of AD and suggest an independent genetic predisposition from the metabolic one.

These allelic variations are associated with measurable differences in IL-10 and IL-6 production by antigen- and mitogen-stimulated peripheral blood lymphocytes. In fact, these polymorphisms occur in the regulatory region of the gene and are associated with high, intermediate or low IL-10 production (22).

The present inventors investigated beta amyloid-stimulated IL-10 and IL-6 production by peripheral blood lymphocytes (PBMC) of AD patients and of age-matched healthy controls. Because the generation of this cytokine was significantly reduced in AD patients, IL-10 polymorphisms were analysed in these same individuals. Results showed that the high IL-10-producing allele is extremely rare in AD patients.

Specifically, IL-10 genotypes are differently distributed when AD are compared with HC (χ²=16.007; p=0.007). Therefore genotypes corresponding to reduced IL-10 production have a significantly higher distribution amongst AD subjects (table I). The presence of low-IL-10-producing genotypes is associated with a worsened clinical outcome of AD as follows: 1) earlier age at disease onset (Table II); and 2) faster disease progression (MMSE score) (Table III).

TABLE I
IL-10 genotype distribution

| | AD | HC | AD | HC |
|---|---|---|---|---|
| Genotype (c) | n = 47 | n = 25 | % | % |
| GCC/GCC (H) | 1 | 7 | 2 | 28 |
| GCC/ACC (M) | 10 | 9 | 21 | 36 |
| GCC/ATA (M) | 11 | 3 | 23 | 12 |
| ACC/ACC (L) | 8 | 1 | 17 | 4 |
| ACC/ATA (L) | 12 | 4 | 26 | 16 |
| ATA/ATA (L) | 5 | 1 | 11 | 4 |

The frequency of the different genotypes among Alzheimer's disease patients (AD) are statistically different from those of the healthy controls (HC). χ²=16.007, df=5, p=0.007. In the brackets (c) there are the corresponding phenotype high (H), intermediate (M), low (L).

TABLE II
IL-10 genotype distribution and age at onset

| | | | |
|---|---|---|---|
| Genotype | mean | S.D. | SEM |

| | | | |
|---|---|---|---|
| GCC/GCC | 76 | / | / |
| GCC/ACC | 75.00 | 8.57 | 3.03 |
| GCC/ATA | 67.33 | 8.2 | 2.73 |
| ACC/ACC | 76.20 | 8.79 | 3.93 |
| ACC/ATA | 77.17 | 4.07 | 1.66 |
| ATA/ATA | 65.75 | 1.71 | 0.85 |

Correlation between the different genotypes in Alzheimer's disease patients and the age at onset. ANOVA: p=0.042.

TABLE III
IL-10 genotype distribution and MMSE

| | | | |
|---|---|---|---|
| Genotype | mean | S.D. | SEM |

| | | | |
|---|---|---|---|
| GCC/GCC | 18 | | |
| GCC/ACC | 21.75 | 5.5 | 1.94 |
| GCC/ATA | 16.33 | 5.68 | 1.89 |
| ACC/ACC | 10.80 | 7.5 | 3.35 |
| ACC/ATA | 13.83 | 5.19 | 2.12 |
| ATA/ATA | 22.5 | 1.73 | 0.87 |

Correlation between the different genotypes in Alzheimer's disease patients and MMSE ANOVA: p=0.010.

Chronic inflammation is suggested to be involved in the neurodegenerative process characteristic of AD (24, 25); this suggestion stems from both in vivo and ex adjuvantibus criteria. Hence, inflammatory mediators and activated glial cells are observed to be closely associated with neuritic plaques in vivo. Furthermore, recent data indicate that anti-inflammatory therapy could be useful in modulating disease progression (10-12). Despite this vast body of evidence, the biologic correlates of AD are still unclear. To shed light on this problem, attention was focused on IL-10. This cytokine is a pivotal regulatory cytokine involved in many facets of the immune response and is dysregulated in human autoimmune (26), malignant (27-31), and infectious (32-35) diseases. More recently it has been shown that the presence of genetically-determined higher levels of IL-10 secretion is an important component of the genetic background to systemic lupus erythematosus (36) and to the outcome of infectious disease (37). It has also been demonstrated that IL-10 secretion, resulted from in vitro stimulation of human peripheral blood leukocytes with LPS, varies markedly between individuals and that cytokine haplotypes are associated with different secretion levels (38). In addition, differences in IL-10 serum production by cells of patients and of their first-degree family members (37, 39), as well as differences in the distribution of IL-10 alleles, suggested the involvement of the different isoforms of the IL-10 gene as an important quantitative trait loci for human disease in infections (37, 40), autoimmune (26, 36, 41, 42) and malignant diseases (43).

The present inventors initially analyzed LPS-, Flu-, and amyloid peptide-specific IL-2 and IL-10 production by peripheral blood mononuclear cells (PBMC) of AD patients and age matched HC. Results showed that: 1) IL-2 production by PBMC of AD patients and controls was similar in all the conditions measured; and 2) IL-10 generation by LPS- and Flu-stimulated PBMC was comparably similar amongst the two groups of individuals. In contrast, an amyloid-specific immune impairment characterized by a reduced generation of IL-10 was present in AD. The observation that this cytokine imbalance was not seen in mitogen-stimulated PBMC indicates that amyloid-specific immune responses are selectively impaired in AD patients. Additionally, results showing that flu-stimulated proliferation was similar in patients and controls indicates that antigenic processing and presentation in association with HLA class II molecules, and the CD4-HLA class II self-restricted pathway of activation of the immune system (44), are not defective in these patients.

Next polymorphisms were analyzed in the IL-10 gene in the same group of subjects. Results stemming from analysis of the distribution of the IL-10 alleles in this Italian sample of healthy individuals showed a close similarity to those reported for other Caucasian populations (45). In contrast, we observed a significantly higher frequency of the genotypes corresponding to reduced IL-10 production (ACC/ACC, ACC/ATA and ATA/ATA) in AD patients. Thus, an abnormally augmented prevalence of low-IL-10 producing isoforms in the AD population was determined; the phenotypic correlate of these isoforms becomes evident when amyloid-specific immune responses were measured.

Subsequent analyses focused on possible correlations between impaired IL-10 production and the clinical manifestations of AD by verifying whether the presence of low/intermediate IL-10 producing genotypes would be are associated with different disease outcomes. Results confirmed this to be the case. Thus, the presence of the ATA/ATA and of the GCC/ATA genotypes was correlated with an earlier age at disease onset. Additionally, the ACC/ATA and the ACC/ACC (all these are low/intermediate IL-10-producing genotypes) alleles were associated with a more severe cognitive impairment as indicated by a lower MMSE score.

It is interesting to observe that a recent report on Italian centenarians, individuals who—by definition—are less prone to develop age-related diseases, has demonstrated that extreme longevity is associated with a significantly higher frequency of the high IL-10-producing genotypes (46).

IL-10 is known to have potent anti-inflammatory properties (47); a biological scenario could thus be hypothesized in which the reduction of amyloid-specific IL-10 production would favour the triggering of the chronic inflammatory process seen in the progression of AD. These results suggest that an amyloid-specific and IL-10-mediated inhibitory feed-back circuit may be active in non-AD individuals; the rupture of this circuit could be associated with or predictive for the development of AD. Recently, a convincing study showed that an IL-10/pro-inflammatory circuit that revolves around cells of the innate immune system regulates susceptibility to autoimmune diseases (48). These results are expanded by showing that an alteration of this circuit is present in AD patients.

The present inventors have identified polymorphic regions, which polymorphs are indicative of a dysfunction of cytokine production and hence are associated with a predisposition towards an autoimmune, neurodegenerative or chronic inflammatory disease.

At present, Alzheimer's disease is diagnosed by recognised criteria such as DMS IV or NINCDS-ADRDA (23), often in conjunction with a magnetic resonance image (MRI) or computer aided tomography (CT) scan of the brain to identify the characteristic amyloid plaques and neurofibrillary tangles together with atrophy of the hippocampal area of the brain.

A definitive confirmatory diagnosis of Alzheimer's disease is only possible by a visual inspection of the affected areas of the brain during a post-mortem examination or via brain biopsy (not recommended due to lack of effective therapies).

Therapies and methods for monitoring of Alzheimer's disease are being urgently sought. As the progress is made in efforts to prevent or delay neurodegeneration and disease progression, early detection of Alzheimer's and identification of susceptible patients will gain importance as this will allow preventive measures being employed as early as possible. Therefore a need exists to be able to provide predictive and reliable tests for susceptibility to Alzheimer's disease without the need for lengthy and subjective assessments of cognitive performance.

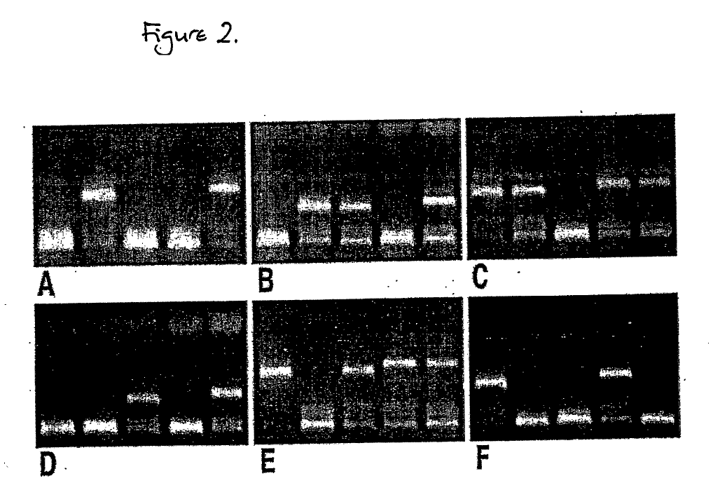

Accordingly, the present invention provides a method of determining the existence of or a predisposition to Alzheimer's disease, autoimmune disease or other neurodegenerative diseases, the method comprising the steps of taking a DNA bearing sample from a subject animal and analysing the sample to determine the allelic variants present at one or more of the SNP loci at positions −1082, −819 and −592 of the gene encoding IL-10, or to put it another way, analysing the sample for the presence or absence of the alleles of FIG. 2.

Preferably, the genotype at all three positions −1082, −819 and −592 is determined.

While the identification of the alleles of FIG. 2 has been found to be useful or predictive in the identification of Alzheimer's disease, a combination of the alleles of IL-10 and IL-6 has been found to be more strongly predictive of a predisposition to Alzheimer's or diagnostic of the presence of Alzheimer's disease.

Apolipoprotein E (Apo-E) has been associated with sporadic or non-familial AD. Hence, in a further aspect of the invention, a method of diagnosing Alzheimer's disease comprises the steps of obtaining a DNA-bearing sample from an animal and identifying the presence of a polymorphic allele of IL-10, IL-6 and of Apo-E.

Preferably, the polymorphic allele is one of the alleles of FIG. 2.

Additionally, the sample may be assayed for the presence/absence of polymorphisms or other allelic variations of other cytokines in addition to IL-10 and IL-6, for example, IL-10 and IL-6 plus IL-4 and/or IL-1.

Alternatively, the sample may be assayed for the presence of absence of polymorphisms or other allelic variations of IL-10 plus Apo-E, or IL-6 plus Apo-E.

An interleukin 1 alpha (IL-1 alpha) polymorphism has been associated with Alzheimer's disease (77). Hence, in still further aspect of the invention, a method of diagnosing Alzheimer's disease comprises the steps of obtaining a DNA-bearing sample from an animal and identifying the presence of a polymorphic allele of IL-10, IL-6, Apo-E and of IL-1.

Generally, optimal predictive value will be obtained by combining as many predictive factors as possible in the test. The methods described herein together with markers such as Apo-E and IL-1 enable the development of a powerful diagnostic method that would include all the biological markers shown to have a predictive value toward the development of AD.

The invention also provides a method of treating Alzheimer's disease, autoimmune diseases or other neurodegenerative disorders by modulating, that is augmenting or decreasing, the function of a gene having one of the allelic polymorphisms of IL-10 shown in Table I, or to put it another way, a gene of the allelic polymorphisms of FIG. 2.

For example, IL-6 production is preferably downregulated but IL-10 production is preferably upregulated. More preferably, IL-6 production is downregulated simultaneously with IL-10 production being upregulated.

Alternatively, pharmaceutical compositions which inhibit or supply the appropriate cytokines may be administered to a patient in need of treatment. For example, instead of down regulation of IL-6 at a genetic level, a patient may be supplied with compounds which inhibit or block the action of IL-6. This inhibition or blocking may be at the synthesis stage, at the site of action or anywhere along the IL-6 metabolic pathway. Similarly, IL-10 may be supplied directly, as an intermediate, as a pre-cursor or pre-pro-cursor, by stimulating the synthesis of IL-10 ab initio or by administration of pharmacological compositions that enhance or inhibit antigen specific production of interleukin-10 and, optionally, one or more other cytokines.

The other cytokine is preferably selected from the group consisting of interleukin-1 (α or β), interleukin-2, interleukin-3, interleukin-4, interleukin-5, interleukin-6, interleukin-7, interleukin-8, interleukin-9, interleukin-11, interleukin-12, interleukin-13, interleukin-14, interleukin-15, interleukin-16, interleukin-17, interferon-α, interferon-β, interferon-γ, TNF-α, TNF-β, G-CSF, GM-CSF, M-LSF, and TGF-β.

Pharmacological agents which can modulate cytokine production are known in the art, for example, heat shock protein (HSP) and/or CpG-motif containing immunomodulatory oligonucleotides. DNA vaccination with constructs encoding the 60-kDa heat shock protein human hsp60 (phsp60) results in increased IL-10 production (71). It has been shown that CpG-DNA can induce the synthesis of suppressor of cytokine signalling (SOC) proteins. GpG-DNA-induced SOC proteins inhibit IL-6 production (72). Additionally, CpG-DNA via the extracellular signal-regulated kinase (ERK) mediated pathway, has been shown to trigger IL-10 production (73). CpG oligonuclotides can be structurally modified to achieve a desired profile of cell types affected and cytokines stimulated; to lean either toward the Th1 (cell mediated, interferon gamma generating) or Th2 (antibody, IL-10 and Il-4 generating) T helper cell pathway (74). Examples of such diverse modulations are: Th1 profiled compound 7909 generated by Coley Pharmaceuticals and Th2 profiled compounds generated by Dynavax (75). In addition, CpG-like immunomodulatory oligonuclotides in which CpG motif has been substituted with YpG or CpR motifs but which show promise of modification of their immunomodulatory potential via their chemical structure may also be employed as pharmacological agents to affect desired cytokine production profile (76).

In a further aspect, the present invention provides a method of treating Alzheimer's disease in an animal in need of treatment, the method comprising the reduction of IL-6 synthesis simultaneously with the augmentation of IL-10 synthesis.

The invention also provides the use of IL-6 inhibitors and IL-10 promoters in the manufacture of a medicament for the treatment of prophylaxis of Alzheimer's disease.

In a further aspect of the invention DNA fragments and cDNA fragments encoding the allelic polymorphism of Table I, or to put it another way the allelic polymorphisms of FIG. 2, for use in the above described method.

These DNA fragments are useful in the screening and identification of compounds which bind to, regulate, or otherwise have a modulatory effect these alleles and hence stimulate or inhibit the synthesis of the gene product.

Accordingly, the present invention further provides a method of screening for compounds which modulate chemokines implicated in Alzheimer's disease, the method comprising introducing the compound to be screened to DNA or cDNA fragments encoding the allelic polymorphisms of Table I, or to put it another way the allelic polymorphisms of FIG. 2 and assessing the hybridisation between the compound and the fragment.

Hence, the present invention also provides compounds which modulate Alzheimer's disease, as identified by the above method.

Preferably, the animal is a mammal and more preferably a human being.

The data presented herein support the role of inflammatory processes in the pathogenesis of AD; reinforce the hypothesis that in AD patients neurodegeneration is tightly associated with an aberrant antigen-specific immune response; and lend further support to the use of anti-inflammatory compounds in the therapy of this disease.

Accordingly, in a still further aspect the present invention provides a pharmaceutical composition comprising a cytokine in the preparation of a medicament for the treatment or prophylaxis of disease excluding neoplastic diseases, leukaemias, and acute inflammation. Preferably the disease is a neurodegenerative disorder or an autoimmune disease. Most preferably the disease is selected from the group comprising multiple sclerosis, myasthenia gravis, systemic lupus erythramatosus, diabetes mellitus, asthma, Parkinson's disease, motor neurone disease, Alzheimer's disease, chronic inflammation rheumatoid arthritis, HIV-infection and AIDS.

Preferably, the cytokine is selected from the group consisting of interleukin-1 (α or β), interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-15, interleukin-16, interleukin-17, interferon-α, interferon-β, interferon-γ, TNF-α, TNF-β, G-CSF, GM-CSF, M-LSF, and TGF-β, or combinations or mixtures thereof. Preferably, two or more cytokines are used.

Most preferably the or each cytokine is an interleukin, especially interleukin-10 or interleukin-6.

Embodiments of the invention will now be described by way of example only, with reference to the accompanying drawings of which

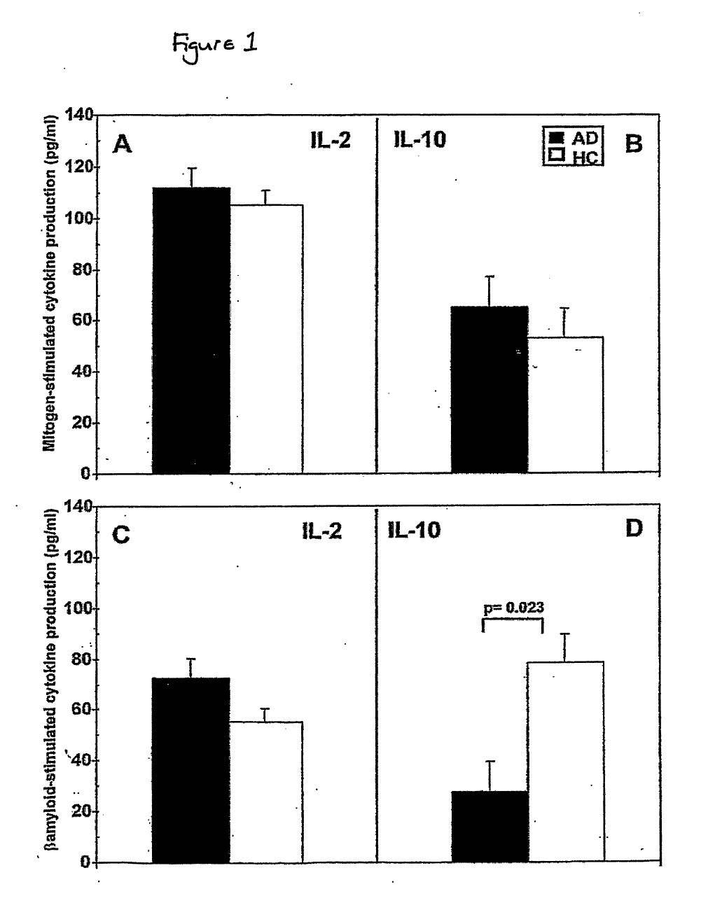

FIGS. 1A-1D are bar charts in which show LPS- and βamyloid—(a pool of 3β amyloid peptides: βA: fragments 25-35; βA: fragment 1-40; and βC: fragment 1-16) stimulated IL-2 (panels A and C) and IL-10 (panels B and D) production by PBMC of 47 AD patients (O) and 25 age- and sex-matched healthy controls (O). Mean values+standard errors are shown. p≦0.023;

FIG. 2 shows paradigmatic example of IL-10 genotyping for six different samples. In each gel the heaviest bands correspond to the amplicons of the human β-globin gene which is used as the internal controls. The other specific amplified DNA fragments correspond to the polymorphisms of the IL-10 gene: GCC/GCC (A), GCC/ACC (B), GCC/ATA (C), ACC/ACC (D), ACC/ATA (E), ATA/ATA (F), and

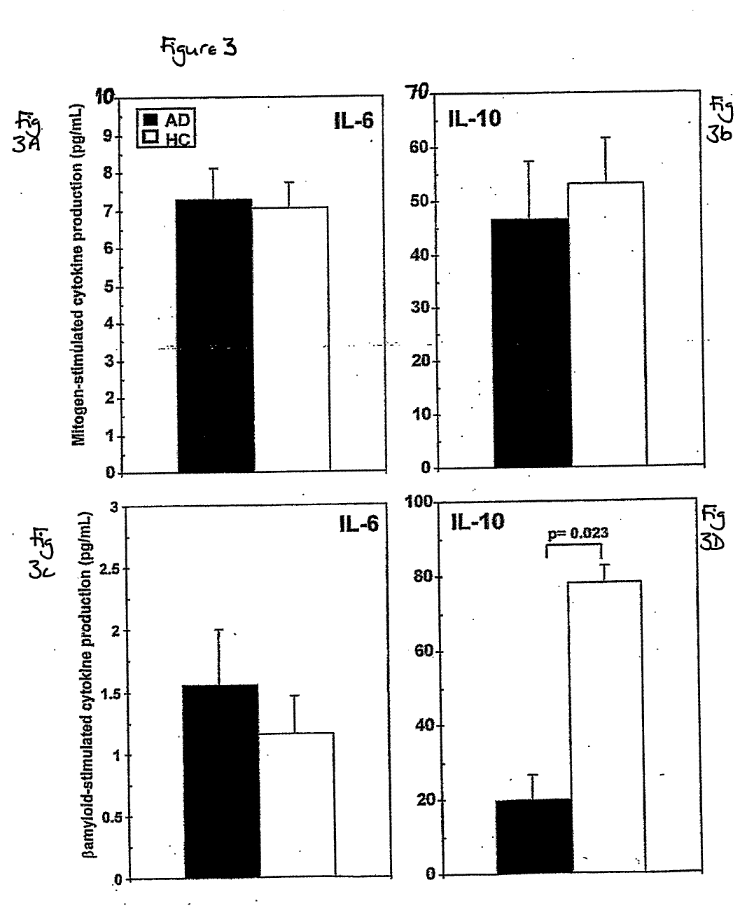

FIGS. 3A-3D are bar charts which show LPS- and β-amyloid-stimulated (a pool of three β-amyloid peptides; βA, fragment 25-35; βB, fragment 1-40; and βC, fragment 1-16) production of IL-6 (panels A and C) and IL-10 (panels B and D) by PBMC of 47 AD patients (O) and 25 age- and sex-matched healthy controls (O). Means+standard errors; p≦0.023.

### EXAMPLE 1

Patients and Controls

Forty-seven AD patients and 25 non-demented subjects (HC) were included in a study of Alzheimer's disease. These patients were selected from a larger population sample followed at the Geriatric Department of the Ospedale Maggiore IRCCS, University of Milan, Italy. The DMS IV and NINCDS-ADRDA (23) criteria were adopted to obtain the clinical diagnosis of AD. Cognitive performances and alterations were assessed according to the Mini-Mental State Evaluation (MMSE). AD patients and HC were living at home and were carefully physical examined on the day of blood collection and their clinical records evaluated. In order to minimize the risk of clinical or sub-clinical inflammatory processes, all the patients were selected as follows: only AD and HC without clinical sign of inflammation (e.g. normal body temperature or absence of concomitant inflammatory disease) were included in the study. Blood chemical parameters were also evaluated and subjects with abnormal sedimentation rate of red blood cells or altered blood profile of albumin and transferring plasma levels were excluded. A further selection of AD patients were performed according to the C reactive protein (CRP) plasma levels and those patients with CRP above 5 mg/l (mean value±2 standard deviations of control values) were not enrolled in the study.

Informed consent to perform the study was obtained from controls and a relative of each AD patient.

Blood Sample Collection

Whole blood was collected by venipuncture in Vacutainer tubes containing EDTA (Becton Dickinson Co, Rutherford, N.J.). Peripheral blood mononuclear cells (PBMC) were separated by centrifugation on lymphocyte separation medium (Organon Teknika Corp., Durham, N.C.) and washed twice in PBS. The number of viable lymphocytes was determined by trypan blue exclusion and a hemocytometer.

In Vitro Cytokine Production

PBMCs were resuspended at 3×10⁶/ml in RPMI 1640 and were either unstimulated or stimulated with LPS (Sigma, St. Louis, Mich.) (10 g/ml), with a pool of 3 different peptides from the b-amyloid protein as follows: b-A: fragment 25-35 (25 mg/ml); b-B: fragment 1-40 (150 ng/ml); b-C: fragment 1-16 (150 ng/ml) (Sigma, St. Louis, Mich.); or with influenza virus vaccine (A/Taiwan+A/Shanghai+B/Victoria) (24 g/l; final dilution 1:1000) (Flu) (control antigen) at 37° C. in a moist, 7% CO₂ atmosphere. Supernatants were harvested after 48 hours for LPS stimulation and after 5 days of culture for the b-amyloid protein peptides and Flu. Production of IL-2 and IL-10 by PBMCs was evaluated with commercial available ELISA kits (ACCUCYTE, Cytimmune Sciences, Inc, College Park, Md.). All test kits were used following the procedures suggested by the manufacturer.

IL-10 Genotyping

Genomic DNA was extracted from EDTA-treated peripheral blood (10 ml) using a standard proteinase K and phenol/chloroform method. The DNA concentration and purity were determined by spectrophotometric analysis. A polymerase chain reaction-sequence specific primers (PCR-SSP) methodology was utilised to assess the IL-10 genotypes. The amplification of the sequence in the promoter region of the IL-10 (polymorphic positions −1082, −819, −592) gene were performed using the Cytokine genotyping Tray Method (One Lambda, Canoga Park, Calif., USA); the human β-globin gene was amplified as an internal control of genomic DNA preparation. PCR condition were indicated by One Lambda PCR program (OLI-1); the PCR products were then visualised by electrophoresis in 2.5% agarose gel.

Statistical Analysis

Statistical analysis was conducted using SPSS statistical package (SPSS, Chicago, Ill.). Differences in IL-10 production stemmed from analytic procedures based on non parametric analyses (Mann-Whitney); comparisons between different groups of patients were made using Fisher's exact 2-tailed test. Genotype frequencies were compared between the study groups by c² test with an observed significance level of the test below 0.05. Comparisons between the mean values of the age at onset and MMSE in the six different groups of AD were performed by one-way ANOVA analysis.

Age, Gender and MMSE Scores in AD Patients and in HC

Forty-seven AD patients and 25 age-matched healthy controls were enrolled in the study. The Mini-Mental State Evaluation (MMSE) showed the presence of a mild-to-severe cognitive deterioration in the AD patients. These data are shown in Table I.

MBP-Stimulated IL-10 Production is Reduced in AD Patients

PBMC of 47 AD patients and of 25 age- and sex-matched HC were stimulated with a mitogen (LPS); a pool of 3 amyloid peptides (A: fragment 25-35, B: fragment 1-40, and C: fragment 1-16) (amyloid), or Flu (used as a control antigen) and the production of IL-2 and IL-10 was measured with ELISA methods. No differences were seen when LPS- or Flu-stimulated IL-2 and IL-10 production was compared in AD patients and HC. amyloid-stimulated IL-2-production was also similar in the two groups of individuals studied. In contrast with these results, amyloid-stimulated production of IL-10 was significantly reduced (p=0.023) in AD patients compared to controls. These data are shown in FIG. 1.

The Distribution of High, Intermediate, and Low IL-10 Producing Genotypes is Skewed in AD Patients

Paradigmatic example of the six different IL-10 genotypes, evaluated by PCR-SSP, is showed in FIG. 2 and their relative distribution among a typical Caucasian population sample is shown in Table II. In contrast with the distribution observed in HC, the frequency of the different IL-10 genotypes among AD patients was significantly skewed (c²=16.007 with p=0.007) (Table II). Therefore genotypes corresponding to reduced IL-10 production (ACC/ACC, ACC/ATA and ATA/ATA genotypes) had a significantly higher distribution amongst AD subjects (17%, 26% and 11% respectively versus 4%, 16% and 4% in HC). Moreover the GCC/ACC to GCC/ATA ratio (intermediate phenotype) was 1:1 in AD while was 3:1 in HC.

Low IL-10 Production is Correlated with Worsened Clinical Outcome of AD

To analyse possible clinical correlates of the presence of low IL-10 genotype, we subsequently examined the six genotypes in relation to age of AD onset (Table III) and the progression of cognitive deterioration (Table IV). The results confirmed that the presence of low-IL-10-producing genotypes is indeed associated with a worsened clinical outcome of AD. Thus, presence of the ATA/ATA and GCC/ATA genotypes was associated with an earlier age at disease onset (ANOVA: p=0.042) (Table III); additionally, an inverse correlation was detected between ACC/ATA and ACC/ACC, low IL-10-producing genotypes, and the MMSE score (ANOVA: p=0.010) (Table IV).

TABLE IV
Genetic Association Data for Autoimmune/Inflammatory Disease
www.grc.nia.nih.gov/branches/rrb/dna/geneticdata.htm

| Chrom | CH-band | Gene | Disease | Allele | P-value | Reference | PubMedID |
|---|---|---|---|---|---|---|---|
| 1 | 1q31.1 | CD45 | Ms | C to G in position 77 of | P = 1.510−4 | Jacobsen M 00 | 11101853 |
| | | | | PTPRC exon 4. | | | |
| 1 | 1q31.1 | CD45 | SCId | deletion | na | Kung C 00 | 10700239 |
| | Mouse | CD45 | autoimmune | glutamate 613 to arginine | na | Majeti R 00 | 11163182 |
| | | | nephritis | | | | |
| 1 | 1q32.1 | IL10 | SLE | −4 kb to 5′ | P = .0001 | Gibson AW 01 | 11238636 |
| 1 | 1q32.1 | IL10 | SS | −10 GCC haplotype (G −1082, | P = <0.05 | Hulkkonen J 01 | 11212157 |
| | | | | C −819, and C −592 | | | |
| | | | | of the IL-10 gene | | | |
| 1 | 1q32.1 | IL10 | RA | genotype −1082GG | P = <0.03 | Huizinga TW 00 | 11085795 |
| 1 | 1q32.1 | IL10 | RA | ATA haplotype, pts w/>4 joints | P = 0.02 | Crawley E 99 | 10366102 |
| 1 | 1q32.1 | IL10 | GVHD | IL-10 (−)1064 | P = <.001 | Middleton PG 98 | 9808588 |
| 1 | 1q32.1 | IL10 | IBD/UC | −1082*G allele (high producer) | P = 0.03 | Tagore A 99 | 10551422 |
| | | | | was reduced in pts | | | |
| 2 | 2q12.2 | IL1RA | SLE | IL1RN*2 allele | na | Blakemore AL 94 | 7945503 |
| 2 | 2q12.2 | IL1RA | Ulcerative | IL1RN*2 allele | P = 0.007 | Mansfield JC 94 | 8119534 |
| | | | Colitis | | | | |
| 2 | 2q12.2 | IL1RA | polymyalagia | IL1RN*2 allele | na | Boiardi L 00 | 11138328 |
| | | | rheumatica | | | | |
| 2 | 2q33.1 | CTLA4 | RA | A/G 49 | P = 0.009 | Gonzalez MF 99 | 10203024 |
| 2 | 2q33.1 | CTLA4 | GD | A/G 49 | P = <0.01 | Yanagawa T 97 | 9459626 |
| 2 | 2q33.1 | CTLA4 | MS | A/G 49 | P = 0.006 | Harbo HF 99 | 10082437 |
| 2 | 2q33.1 | CTLA4 | H-Thy | A/G 49 | P = <0.03 | Donner H 97 | 9398726 |
| 2 | 2q33.1 | CTLA4 | IDDM | A/G 49 | P = 0.004 | Takahiro A 99 | |
| 2 | 2q33.1 | CTLA4 | IDDM | | na | Yanagawa T 99 | 10052685 |
| 2 | 2q33.1 | CTLA4 | IDDM | A/G 49 | P = 0.00002 | Marron MP 97 | 9259273 |
| 5 | 5q31.1 | IL4 | GD | position 590 allele reduced | P = 0.00004 | Hunt PJ 00 | 10843185 |
| | | | | in GD | | | |
| 5 | 5q31.1 | IL4 | increased IgE | C + 33T polymorphism with | P = <0.05 | Suzuki I 00 | 11122213 |
| | | | | elevated total serum IgE | | | |
| 5 | 5q31.1 | IL4 | asthma, FEV(1) | C−589T IL-4 promoter | P = 0.013 | Burchard EG 99 | 10471619 |
| | | | | genotype (TT) | | | |
| 5 | 5q31.1 | IL4 | AD | −590C/T | P = 0.001 | Kawashima T 98 | 9643293 |
| 5 | 5q31.1 | IL4 | RA | IL-4(2) higher in non- | P = 0.0006 | Buchs N 00 | 11035134 |
| | | | | destructive RA | | | |
| 5 | 5q31.1 | IL4 | MS | IL-4 B1 allele, late | P = <0.001 | Vandenbroeck K 97 | 9184650 |
| | | | | onset MS | | | |
| 5 | 5q31.1 | IL13 | asthma | Gln110Arg | P = 0.017 | Heinzmann A 00 | 10699178 |
| 5 | 5q31.1 | IL13 | asthma | C toT at position - | P = 0.002 | van der Pouw | 11197307 |
| | | | | 1055 (TT) | | Kraan TC 99 | |
| 6 | 6p21.31 | TNFa | asthma | G/A −308 TNF2 | P = 0.003 | Albuquerque R 98 | 9645594 |
| 6 | 6p21.31 | TNFa | PrimBilCirr | G/A −308 TNF1 | P = 0.02 | Gordon M 99 | 10453936 |
| 6 | 6p21.31 | TNFa | Sepsis | G/A −308 TNF2 | P = 0.007 | Majetschak M 99 | 10450735 |
| 6 | 6p21.31 | TNFa | Psoriasis | G/A −308 TNF1 | P = 2.74 × 10−8 | Arias A 97 | 9395887 |
| 6 | 6p21.31 | TNFa | lep. Leprosy | G/A −308 | P = .02 | Roy S 97 | 9237725 |
| 6 | 6p21.31 | TNFa | GVHD | TNFd | P = .006 | Middleton PG 98 | 9808588 |
| 6 | 6p21.31 | TNFa | Silicosis | G/A −308 TNF1 | P = <0.05 | Yucesoy B 01 | 11264025 |
| 6 | 6p21.31 | TNFa | SLE | G/A −308 TNF1 | na | Sullivan KE 97 | 9416858 |
| 6 | 6p21.31 | TNFa | celiac | G/A −308 TNF1 | P = <0.001 | McManus R 96 | 8655356 |
| 6 | 6p21.31 | TNFa | chronic | G/A −308 TNF1 | P = <0.01 | Huang S 97 | 9372657 |
| | | | bronchitis | | | | |
| 6 | 6p21.31 | TNFa | Psoriasis | −238 TNF1 | P = 1.64 × 10−7 | Arias A 97 | 9395887 |
| 7 | 7p15.3 | IL6 | IDDM | G,G(−174) increased | P = <0.002 | Jahromi MM 00 | 11054276 |
| | | | | in pts | | | |
| 7 | 7p15.3 | IL6 | SLE | AT-rich minisatellite in | P = 0.001 | Linker-Israeli M 99 | 11197305 |
| | | | | 3′ flanking region | | | |
| 7 | 7p15.3 | IL6 | RA | 622 and −174 alleles, | na | Pascual M 00 | 11196696 |
| | | | | age of onset | | | |
| 7 | 7p15.3 | IL6 | MS | carriage larger alleles | P = 0.025 | | |
| | | | | A6-->A9, accelerated onset | | | |
| 12 | 12q12 | VDR | GD | exon 2 initiation codon | P = 0.023 | Ban Y 00 | 11134121 |
| | | | | (VDR-FOK: I) polymorphism | | | |
| 12 | 12q12 | VDR | RA | BB/tt genotype | na | Garcia-Lozano JR | 11251690 |
| | | | | | | 01 | |
| 12 | 12q12 | VDR | MS | bb | P = 0.0263 | Fukazawa T 00 | 10465499 |
| 12 | 12q12 | VDR | CD | tt | P = 0.017 | Simmons JD 00 | 10896912 |
| 12 | 12q12 | VDR | IDDM | Bsml | P = 0.015 | Chang TJ 00 | 10792336 |
| 12 | 12q21.1 | IFNG | asthma | CA repeat polymorphism | P = .0018 | Nakao F 01 | 11240951 |
| | | | | within the first intron | | | |
| 12 | 12q21.1 | IFNG | IDDM | CA repeat polymorphism | P = 0.039 | Awata T 94 | 7867888 |
| | | | | within the first intron | | | |
| 12 | 12q21.1 | IFNG | GD | CA repeat polymorphism | P = <0.04 | Siegmund T 98 | 9848715 |
| | | | | within the first intron | | | |
| 12 | 12q21.1 | IFNG | RA | CA repeat polymorphism | P = <0.0001 | Khani-Hanjani A 00 | 11022930 |
| | | | | within the first intron | | | |
| 16 | 16p11.1 | IL4R | asthma | Ile50Val | P = <0.0001 | Mitsuyasu H 98 | 9620765 |
| 16 | 16p11.1 | IL4R | hyper-IgE | Arg576G | P = 0.001 | Hershey GKK 97 | 9392697 |
| | | | syndrome and | | | | |
| | | | severe eczema, | | | | |
| | | | atopy | | | | |
| 16 | 16p11.1 | IL4R | MS(PPMS) | IL4R variant R551 | P = 0.001 | Hackstein H 01 | 11164908 |

### [DNA Array Unit] [IRP Home] [NIA Home]

### EXAMPLE 2

Patients and Controls

Sixty-five AD patients (44 F/21 M, mean age 80±2) and 65 non-demented sex- and age-matched healthy controls (HC) were enrolled. The patients were selected from a larger population sample followed at the Geriatric Department of the Ospedale Maggiore IRCCS, University of Milan, Italy. We applied the DMS IV and NINCDS-ADRDA (23) criteria to obtain the clinical diagnosis of AD; every subject had a recent brain magnetic resonance imaging (MRI)/computed tomography (CT) scan available. Cognitive performances and alterations were assessed according to the Mini-Mental State Evaluation (MMSE). AD patients and HC were living at home and a careful physical examination was done on the day of blood collection, and their clinical records were consulted.

In order to minimize the risk of clinical or sub-clinical inflammatory processes, subjects were selected as follows: only AD and HC without clinical signs of inflammation (e.g. normal body temperature, no concomitant inflammatory condition) were eligible. Blood chemistry tests were done and subjects with an abnormal red blood cell sedimentation rate or altered albumin and transferring plasma levels were excluded. AD patients were further selected according to their C reactive protein (CRP) plasma levels and any with CRP above 5 mg/L (mean+2 standard deviations of control values) were not eligible.

Informed consent was obtained from all the subjects or their relatives. The study protocol was approved by the Ethics Committee of the University Hospital.

Blood Sampling

Whole blood was collected by venipuncture in Vacutainer tubes containing EDTA (Becton Dickinson Co., Rutherford, N.J.). Peripheral blood mononuclear cells (PBMC) were separated by centrifugation on lymphocyte separation medium (Organon Teknika Corp., Durham, N.C.) and washed twice in PBS. Viable lymphocytes were counted by Trypan blue exclusion and a hemocytometer.

Genotyping

Genomic DNA was extracted using a standard proteinase K and phenol/chloroform method. The DNA concentration and purity were determined by spectrophotometric analysis. A polymerase chain reaction-sequence-specific primers (PCR-SSP) method was utilised to assess IL-10 and IL-6 genotypes. The sequence in the promoter region of the IL-10 (polymorphic positions −1082, −819, −592) and IL-6 (polymorphic position −174) genes was amplified using the cytokine genotyping tray method (One Lambda, Canoga Park, Calif., USA); the human β-globin gene was amplified as an internal control for the genomic DNA preparation. PCR conditions were indicated by the One Lambda PCR program (OLI-1) and the PCR products were visualised by electrophoresis in 2.5% agarose gel.

The ApoE genotypes were determined by PCR amplification of a 234 base-pair fragment of exon 4 of the ApoE gene, followed by digestion with Cfo1. The restriction patterns were obtained by gel electrophoresis.

In Vitro Cytokine Production

PBMCs were resuspended at 3×10⁶/mL in RPMI 1640 and were either unstimulated or stimulated with LPS (Sigma, St. Louis, Mich.) (10 μg/mL), with a pool of three peptides from the β-amyloid protein as follows: β-A, fragment 25-35 (25 g/mL); β-B, fragment 1-40 (150 ng/mL); β-C, fragment 1-16 (150 ng/mL) (Sigma), or with influenza virus vaccine (A/Taiwan+A/Shanghai+B/Victoria) (24 μg/L; final dilution 1:1000) (Flu) (control antigen) at 37° C. in a moist, 7% CO₂ atmosphere. Supernatants were harvested after 48 hours for LPS stimulation and after five days of culture for the β-amyloid protein peptides. Production of IL-10 and IL-6 by PBMCs was evaluated with commercial ELISA kits (ACCUCYTE, Cytimmune Sciences Inc., College Park, Md.). All test kits were used following the manufacturer's directions.

Statistical Analysis

Statistical analysis was done using the SPSS statistical package (SPSS, Chicago, Ill.). Genotype frequencies were compared in the study groups by the χ² test with a level of significance below 0.05. The odds ratio (OR) and 95% confidence intervals (CI) were also calculated. Adjusted ORs were estimated by logistic regression, controlling for ApoE 4 carrier status. Homogeneity of the ORs between strata was assessed by including the appropriate interaction terms in the model. Differences in IL-10 and IL-6 production were established by procedures based on non-parametric analysis (Mann-Whitney); different groups of patients were compared using Fishers exact two-tailed test.

The Distribution of High, Intermediate, and Low IL-10 Producing Genotypes is Skewed in AD Patients

The genotype and allele frequencies of the biallelic polymorphism at position −1082 are reported in Table V. This SNP alters transcriptional activation with a gene dosage-related effect, so GG genotype correlates with high, GA with intermediate and M with low IL-10 production after stimulation of T cells in vitro (57). AD patients show a significantly higher frequency of the −1082A low producer allele, which skews the genotype distribution in AD compared to HC with a significant decrease of −1082GG high producer genotype (Table V).

TABLE V
Frequency of the different IL-10 genotypes and alleles observed in
Alzheimer's disease patients (AD) and in healthy age-matched controls.

| | Genotype | | | Allele | |
|---|---|---|---|---|---|
| | G/G (H)^{a} | G/A (M) | A/A (L) | A | G |
| AD | 4 (6.4%) | 28 (44.4%) | 31 (49.2%) | 90 (71.4%) | 36 (28.6%) |
| HC | 14 (22.2%) | 29 (46%) | 20 (31.8%) | 69 (54.8%) | 57 (45.2%) |

^{a}The corresponding phenotypes: high (H), intermediate (M), low (L) are shown in brackets
Genotype: χ² = 7.946, df = 2, p = 0.019
Allele: χ² = 6.817, df = 1, p = 0.009

The some SNP is linked with two other SNPs at positions −819 and −592. They combine with microsatellite alleles to form haplotypes where the difference in IL-10 production is mainly accounted for by the −1082 SNP (38, 42). The genotype and allele frequencies of −819 C→T and −592 C→A SNPs were distributed similarly in our AD and HC samples (data not shown).

The −174C Allele in the IL-6 Gene is Over-Represented in AD Patients

The distribution of IL-6 genotypes and alleles in HC and AD is shown in Table 6. This functional polymorphism also seemed related to the plasma IL-6 concentration; however, it is not clear how this SNP influences IL-6 plasma levels (54). The results of the genotype distribution in our AD and HC samples, with a lower frequency of GG genotype in AD patients. Similarly, the allele distribution was significantly different in the two groups, the C allele being significantly higher in AD (Table VI).

TABLE VI
Frequency of the different IL-6 genotypes and alleles observed in
Alzheimer's disease patients (AD) and in healthy age-matched controls.

| | Genotype | | | Allele | |
|---|---|---|---|---|---|
| | G/G (H)^{a} | G/C (H) | C/C (L) | C | G |
| AD | 17 (29%) | 34 (57.6%) | 8 (13.4%) | 50 (42.4%) | 68 (57.6%) |
| HC | 32 (50%) | 27 (42.2%) | 5 (7.8%) | 37 (28.9%) | 91 (71.1%) |

^{a}High (H) and low (L) phenotypes are in brackets
Genotype: χ² = 5.894, df = 2, p = 0.052
Allele: χ² = 4.300, df = 1, p = 0.038

L-10 and IL-6 Allele Combination and Relative Risk of Developing AD

We investigated whether any combination of the IL-10 GA and IL-6 GC alleles affected the risk of AD. The concomitant presence of both IL-10 A and IL-6 C alleles significantly raised this risk, independently of the ApoE4 status (Table VII). The IL-10 A/A genotype alone or the IL-6 C/C genotype alone both conferred a smaller increase in the risk of the disease (OR 5.8, CI 1.7-20, p=0.005; OR 3.0, CI 0.9-10.6, p=0.087).

TABLE VII
IL-10 and IL-6 alleles and risk for Alzheimer disease

| IL-10 | IL-6 | OR | 95% CI | adj. OR | 95% CI |
|---|---|---|---|---|---|
| G allele | G allele | 1 | | 1 | |
| G | C | 2.8 | 0.2-40 | 0.9 | 0.1-26.5 |
| A | G | 4.6 | 0.5-41 | 3.3 | 0.3-36.3 |
| A | C | 11.2* | 1.3-97.3 | 10.3* | 1.0-108 |

7*p > 0.05;
OR: crude odds ratio;
adj. OR: apolipoprotein E ε4 adjusted odds ratio;
CI: confidence interval

LPS, Flu, and Amyloid Peptide-Stimulated IL-10 and IL-6 Production is Reduced in AD Patients

PBMC of 47 AD patients and 25 age- and sex-matched HC were stimulated with a mitogen (LPS), with a pool of three β amyloid peptides (βA, fragment 25-35; βB, fragment 1-40; βC, fragment 1-16), or with Flu and the production of IL-10, IL-6 was measured with ELISA methods. There were no differences in LPS- or flu-stimulated IL-6 and IL-10 production in AD and HC. In contrast, when β-amyloid-stimulated production of IL-6 and IL-10 was analysed, a marginal increased IL-6 production and a significant decrement of IL-10 generation (p=0.023) were seen in AD patients compared to HC, suggesting an antigen-specific impairment in the production of these cytokines. These data are shown in FIG. 3.

The causative role of chronic inflammation in the pathogenesis of AD is still mainly speculative (24, 25). Nonetheless a “cytokine cycle” has been proposed where (19) the anti-inflammatory cytokines (IL-4, IL-10 and IL-13) regulate β-amyloid-induced microglial/macrophage inflammatory responses and modify the microglial activity surrounding amyloid neuritic plaques (52). These cytokines can inhibit the induction of IL-1, TNF-α and MCP-1 in differentiated human monocytes and, above all, IL-10 causes dose-dependent inhibition of the IL-6 secretion induced by β-amyloid in these cells and in murine microglia (19).

From a clinical point of view, IL-10 is involved in autoimmune diseases (41, 42, 26) and in malignancies (31, 27, 43) where the higher levels of the cytokine depend on genetic background (59) but also influence the outcome of infections (34, 40, 37).

More consistent is the evidence of a role of IL-6 in the pathogenesis of AD. Elevated IL-6 immunoreactivity was observed close to amyloid plaques in the brain of these patients (67); IL-6 induces the synthesis of β-amyloid precursor protein (69), and in transgenic mouse models elevated CNS levels of IL-6 result in neuropathogenic effects and cognitive deficits (51).

The C allele of a VNTR on the IL-6 gene was reported to reduce cytokine activity (61). The IL-6 VNTR C allele has been correlated with a delayed initial onset and reduced AD risk in a German population (63). The functional polymorphism −178 of the promoter region could also be involved in the development of AD phenotype because of its association with plasma concentrations of the cytokine (54). However, in two clinical sets of different ethnic origin the results were debatable (49).

In our sample the data from SNP analysis showed HC had a distribution of IL-10 and IL-6 alleles similar to that of an Italian population (65). More importantly, the present results point to a significantly higher percentage of IL-10 −1082A carriers among AD cases. A recent report on Italian centenarians, who are clearly less prone than younger persons to age-related diseases, showed that extreme longevity is significantly associated with the high IL-10-producing genotypes (58).

As we have previously reported, the results on IL-6 SNPs are more contradictory. The IL-6 G allele seems significantly in AD of Japanese (66) and also of southern Italian origin (64), whereas in our sample it is the C allele that appears over-represented.

To link these differing findings several points have to be considered. Ethnicity may strongly influence the role of genetic risk factors, and so may the distribution of gene variants in the populations of different European countries, or even among different areas of the same country (53, 55, 60, 62, 70). In addition, the association between AD and IL-6 SNPs may be confined to particular ages, and in our samples AD and HC subjects were all old-old.

Finally, we must considered the role played by a gene or by several genes in linkage disequilibrium with this mutation: a strong disequilibrium between −174 SNP and the VNTR polymorphism of the 3′ flanking region of the IL-6 gene has been described in Germans (49).

The main finding of this study was the identification of a group of subjects with a high risk of late-onset AD on account of the concomitant presence of IL-10 −1082A and IL-6 −174C alleles. We also explored interactions between Apo E and IL-10 or IL-6 genes but did not find any evidence of synergistic effects, suggesting that these inflammation-related alleles are an additional and independent risk factor for AD.

To shed more light on the genetic results, the inventors also analysed β-amyloid peptide-, LPS-, and Flu-specific IL-10 and IL-6 production by peripheral blood mononuclear cells (PBMC) in a subset of AD patients and age-matched HC. The results showed that: 1) IL-6 production by PBMC of AD patients and controls did not differ significantly in any conditions; and 2) IL-10 generation by LPS- and Flu-stimulated PBMC was comparable in the two groups, whereas a β-amyloid-specific immune impairment characterized by a reduced generation of IL-10 was noted in AD. The fact that this cytokine imbalance was not seen in mitogen-stimulated PBMC indicates that β-amyloid-specific immune responses are selectively impaired in AD. Additionally, the finding that flu-stimulated proliferation was similar in patients and controls indicates that antigenic processing and presentation in association with HLA class II molecules, and the CD4-HLA class II self-restricted pathway of activation of the immune system (44), are not defective in AD. Thus a biological scenario is conceivable in which the reduction of amyloid-specific IL-10 production favours the triggering of the chronic inflammatory process seen in the AD brain. An amyloid-specific and IL-10-mediated inhibitory feedback circuit could be active in non-AD individuals, and a breakdown of this circuit could be associated with, or predictive of, the development of AD. A recent study showed convincingly that an IL-10/pro-inflammatory circuit revolving around cells of the innate immune system regulates susceptibility to autoimmune diseases (48). Our results extend this concept by showing that in AD patients this circuit is altered. The data as a whole support the theory that the overall risk of developing AD may be governed by a “susceptibility profile”, that reflects the combined influence of inheriting multiple high-risk alleles, and casts light on the pivotal role of IL-10 and IL-6 SNPs in this profile.

Inflammation is involved in the pathogenesis of Alzheimer's disease (AD, the anti-inflammatory cytokine interleukin-10 (IL-10) might counteract IL-6 activity in the brain. As the promoter of these genes is polymorphic, the 65 AD patients and 65 healthy controls (HC) the present investigated the IL-10 −1082 GA and IL-6 −174 GC alleles. In several cases they also assessed IL-10 and IL-6 production by PBMC. For IL-10 there was a significant higher level of the −1082GG genotype (p=0.019) in HD than HC, while for IL-6 the G/G genotype was lower and the C allele higher (p<0.005). The concomitance of IL-10 A and IL-6 C alleles significantly raised the risk of AD (odds ratio: OR 11.2, confidence interval: CI 1.3-97.3; p<0.05) independently of ApoE4 (adjusted OR 10.3, CI 1-108; p<0.05). Only amyloid-stimulated IL-10 production differed in AD and HC (p=0.023). These results conflict with the inflammatory theory in AD, pointing to a pivotal role of IL-10 and IL-6 polymorphisms and a selective alternation in this network.

### EXAMPLE 3

Genotype Analyses on Interferon-γ and TNF-α

The methods described in the preceding Examples were used to perform genotype analysis on interferon-γ and TNF-α in Alzheimer's patients and healthy controls. A summary of the results is shown in tables VIII and IX.

TABLE VIII
IFN-γ genotype distribution

| | Genotype (c) | | | Allele | |
|---|---|---|---|---|---|
| | T/T (H) | T/A (I) | A/A (L) | T | A |
| AD | 11(15.5%) | 35(49.3%) | 25(35.2%) | 57(40%) | 85(60%) |
| HC | 11(18%) | 31(51%) | 19(31%) | 53(43%) | 69(57%) |

The frequencies of the different genotypes among Alzheimer's disease patients (AD) were not statistically different from those of the health controls (HC).
χ²=0.305, df=2, p=0.859.
In the brackets (c) there are the corresponding phenotype high (H), intermediate (M) and low (L).

Allele:
χ²=0.174, df=1, p=0.676.

TABLE IX
TNF-α genotype distribution

| | Genotype | | | Allele | |
|---|---|---|---|---|---|
| | G/G (L)^{a} | G/A (H) | A/A (H) | C | G |
| AD | 60 (82%) | 12 (16.5%) | 1 (1.5%) | 132 (90%) | 14 (10%) |
| HC | 32 (69%) | 13 (28%) | 1 (3%) | 77 (84%) | 15 (16%) |

The frequencies of the different genotypes among Alzheimer's disease patients (AD) were not statistically different from those of the health controls (HC)
^{a} High (H) and low (L) phenotypes are in brackets
Genotype: χ²=2.568, df=2, p=0.277
Allele: χ²=1.792, df=1, p=0.181

There are no statistically significant differences when Alzheimer's patients and controls are compared indicating that neither interferon-γ nor TNF-α is associated with the likelihood of developing Alzheimer's disease.

In contrast, the present invention shows that IL-10 and IL-6 are highly predictive for developing Alzheimer and possibly also predict disease progression. The best predictive value will be achieved by combining genotype tests for multiple gene polymorphisms e.g. IL-10, IL-6, Apo-E and others shown to be associated with Alzheimer's disease.

### REFERENCES

1. Ernst, R. L. and J. W. Hay. 1994. The US economic and social costs of Alzheimer's disease revised. Am. J. Public Health. 84: 1261.
2. Goate, A., M. C. Chartier-Harlin, M. Mullan, J. Brown, F. Crawford, L. Fidani, L. Giuffra, A. Haynes, N. Irving, L. James, R. Mant, P. Newton, K. Rook, P. Roques, C. Talbot, M. Pericak-Vance, A. Roses, R. Williamson, M. Rossor, M. Owen and J. Hardy. 1991. Segregation of a missense mutation in the amyloid precursor protein gene with familial Alzheimer's disease. Nature. 349: 704.
3. Levy-Lahad, E., W. Wasco, P. Poorkaj, D. M. Romano, J. Oshima, W. H. Pettingell, C. E. Yu, P. D. Jondro, S. D. Schmidt, K. Wang, A. C. Crowley, Y. H. Fu, S. Y. Guenette, D. Galas, E. Nemens, E. M. Wiisman, T. D. Bird, G. D. Schellemberg and R. E. Tanzi. 1995. Candidate gene for the chromosome 1 familial Alzheimer's disease locus. Science. 269: 973.
4. Sherrington, R., E. I. Rogaev, Y. Liang, E. A. Rogaeva, G. Levesque, M. Uveda, H. Chi, C. Lin, G. Li, K. Holman, T. Tsuda, L. Mar, J. F. Fonci, A. C. Bruni, M. P. Montesi, S. sorbi, I. Rainero, L. Pinessi, L. Nee, I. Chumaken, D. Pollen, A. Brookes, P. Sanseau, R. J. Polinsky, W. Wasco, H. A. R. Da Silva, J. L. Haines, M. A. Pericak-Vance R. E. Tanzi, A. D. Roses, P. E. Fraser, J. M. Rommens and P. H. George-Hyslop. 1995. Cloning of a gene bearing missense mutation in early-onset familial Alzheimer's disease. Nature. 375: 754.
5. Blacker, D., J. L. Haines, L. Rodes, H. Terwedow, R. C. P. Go, L. E. Harrel, R. T. Perry, S. S. Basset, G. Chase, D. Meyers, M. S. Albert and R. Tanzi. 1997. ApoE-4 and age at onset of Alzheimer's disease: The NINH Genetics Initiative. Neurology. 48: 139.
6. Poirier, J., J. Davignon, D. Bouthillier, S. Kogan, p. Bertrand and S. Gauthier. 1993. Apolipoprotein E polymorphism and Alzheimer's disease. Lancet 342: 697.
7. Saunders, A. M., W. J. Strittmatter, D. Schmechel, P. H. George-Hyslop, M. A. Pericak-Vance, S. H. Joo, B. L. Rosi, J. F. Gusella, D. R. Crapper-MacLachlan and M. J. Alberts. 1993. Association of apolipoprotein E allele epsilon 4 with late-onset familial and sporadic Alzheimer's disease. Neurology. 43:1467.
8. Fassbender, K., C. Masters and K. Beyreuther. 2000. Alzheimer's disease: an inflammatory disease? Neurobiology of Aging. 21: 433.
9. McGeer, P. L. and E. G. McGeer. 2001. Inflammation, autotoxicity and Alzheimer disease. Neurobiology of Aging. 22: 799.
10. Zandi, P. P and J. C. S. Breitner. 2001. Do NSAIDs prevent Alzheimer's disease? And, if so, why? The epidemiological evidence. Neurobiology of Aging. 22: 811.
11. Rogers, J., L. C: Kirby, S. R. Hempelman, D. L. Berry, P. L. McGeer, A. W. KasniaK, j. Zalinski, M. Cofield, L. Mansukhani, and P. Willson. 1993. Clinical trial of indomethacin in Alzheimer's disease. Neurology. 43: 1609.
12. Hauss-Wegrzyniak, B., L. B. Willard, P. Del Soldato, G. Pepeu and G. L. Wenk. 1999. Peripheral administration of novel anti-inflammatories can attenuate the effects of chronic inflammation within the CNS. Brain Res. 815: 36.
13. Neuroinflammation Working Group. 2000. Inflammation and Alzheimer's disease. Neurobiology of Aging. 21: 383.
14. Licastro, F., S. Pedrini. L. Caputo, G. Annoni, L. J. Davis, C. Ferri, V. Casadei and L. M. E. Grimaldi. 2000. Increased plasma levels of interleukin-1, interleukin-6 and a-1-antichymotrypsin in patients with Alzheimer's disease: peripheral inflammation or signals from brain? Journal of Neuroimmunology. 103: 97.
15. Mrak, R. E. and W. S. T. Griffin. 2001. Interleukin-1, neuroinflammtion, and Alzheimer's disease. Neurobiology of Aging. 22: 903.
16. Perry, R. T., J. S. Collins, H. Wiener, R. Acton and R. C. P. Go. 2001. The role of TNF and its receptors in Alzheimer's disease. Neurobiology of Aging. 22: 873.
17. Wyss-Coray, T., E. Masliah, M. Mallory, L. McConlogue, K. Johnson-Wood, C. Lin and L. Mucke. 1997. Amyloidogenic role of cytokine TGF-beta1in trangenic mice and in Alzheimer's disease. Nature. 389: 603.
18. Meda, L., P. Baron, E. Prat, E. Scarpini, R. Delgado, A. Catania, J. M. Lipton and G. Scarlato. 1999. Proinflammatory profile of cytokine production by human monocytes and murine microglia stimulated with beta-amyloid. J. Immunol. 93: 45.
19. Szczepanik, A. M., S. Funes, W. Petko and G. E. Ringheim. 2001. Il-4, IL-10 and Il-13 modulate A beta (1-42)-induced cytokine and chemokine production in primary murine microglia and a human monocyte cell line. J. Neuroimmunol. 113: 49.
20. Lombardi, V. R., M. Garcia, L. Rey and R. Cacabelos. 1999. Characterization of cytokine production, screening of lymphocyte subset patterns and in vitro apoptosis in healthy and Alzheimer's Disease (AD). J Immunol. 97: 163.
21. Engelborghs, S., M. De Brabander, J. De Cree, R. D'Hooge, H. Geerts, H. Verhaegen and P. P. De Deyn. 1999. Unchanged levels of interleukins, neopterin, interferon-gamma and tumour necrosis factor-alpha in cerebrospinal fluid of patients with dementia of the Alzheimer type. 34: 523.
22. Hutchinson, I. V., V. Pravica and P. J. Sinnot. 1998. Genetic regulation of cytokine synthesis: consequences or acute and chronic organ allograft rejection. Graft. 1: 15.
23. McKhann, G., D. Drachman, M. Folstein, R. Katzman, D. Proce, E. M. Stadlan. 1984. Clinical diagnosis of Alzheimer's disease. Neurology. 34: 939.
24. Eikelenboom, P., S. S. Zhan, W. A. van Goll and D. Allsop D. 1994. Inflammatory mechanisms in Alzheimer disease. Trends Pharmacol Sci 15:447.
25. Rogers, J., S. Webster, and L. F. Lue. 1996. Inflammation and Alzheimer's disease pathogenesis. Neurobiol Aging 17:686.
26. Llorente, L., W. Zou, Y. Levy, Y. Richaud-Patin, Y. Wijdenes, J. Alcocer-Varela, B. Morel-Fourrier, J. C. Brouet, D. Alarcon-Segovia, P. Galanaud. 1995. Role of interleukin 10 in the B lymphocyte hyperactivity and autoantibody production of human systemic lupus erythemattosus. J. Exp. Med. 181: 839.
27. Luscher, U., L. Filgueira, A. Juretic, M. Zuber, L. J. Luuscher, M. Heberer and g. C. Spagnoli. 1994. The pattern of cytokine gene expression in freshly excised human metastatic melanoma suggests a state of reversible anergy of tumor-infiltrating lymphocytes. Int. J. Cancer. 57: 612.
28. Matsuda, M., F. Slazar, M. Petersson, G. Masucci, J. Hansson, Q. C. Zhang, M. G. Masucci and R. Kiessling. 1994. Interleukin 10 pretreatment protects target cells from tumor- and allo-specific cytottoxic T cells and downregulates HLA class I expression. J. Exp. Med. 180: 2371.
29. Kim, J., R. L. Modlin, R. L. Moy, S. M. Dubinett, T. McHugh, B. J. Nickloff and K. Uyemura. 1995. IL-10 production in cutaneous basal and squamos cell carcinomas. A mechanism for evading the local T cell immune response. J. Immunol. 155: 2240.
30. Suzuki, T., H. Tahara, S. Narula, K. W. Moore, P. D. Robbins, and M. T. Lotze. 1995. Viral interleukin 10 (IL-10), the human herpes virus 4 cellular IL-10 homologue, induces local anergy to allogenic and syngenic tumors. J. Exp. Med. 182: 447.
31. Fortis, C., M. Foppoli, L. Gianotti, L. Galli, G. Citterio, G. Consogno, O. Gentilini and M. Braga. 1196. Increased interleukin-10 serum levels in patients with solid tumours. Cancer Lett. 104: 1.
32. Murray, P. J., L. Wang, R. C. Onufry, R. I. Tepper, and R. A. Young. 1997. T cell-derived IL-10 antagononizes macrophage function in mycobacterial infection. J. Immunol. 158: 315.
33. Lehmann, A. K., A. Halstenen, S. Sornes, O. Rokkeand A. Waage. 1995. High levels of interleukin 10 in serum are associated with fatality in meningococcal disease. Infect Immun. 63: 2109.
34. Clerici, M., T. A. Wynn, J. A. Berzofsky, R. L. Coffman, A. Sher, G. M. Shearer. 1994. Role of Interleukin-10 (IL-10) in T Helper Cell Dysfunction in Asymptomatic Individuals Infected with the Human Immunodeficiency Virus (HIV-1). J. Clin. Invest. 93:768.
35. VanFurth, A. M., E. M. Seijmonsbergen, J. A. M. Langermans, P. H. P. Groeneveld, C. E. Debel and R. VanFurth. 1995. High levels of interleukin 10 and tumor necrosis factor alpha in cerebrospinal fluid during the onset of bacterial meningite. Clin. Infect. Dis. 21: 220.
36. Llorente, L., Y. Richaud-Patin, J. Couderc, D. Alarcon-Segovia, R. Ruiz-Soto, N. Alcocer-Castillejos, J. Alcocer-Varela, J. Granados, S. Bahena, P. Galanaud and D. Emilia. 1997. Dysregulation of interleukin-10 production in relatives of patients with systemic lupus erythematosus. Arthritis Rheum. 38: 1429.
37. Westendorp, R. G. J., J. A. M. Langermans, T. W. G. Huizinga, A. H. Elouali, C. L. Verwej and J. P. Vandenbroucke. 1997. Genetic influence on cytokine production and fatal meningococcus disease. Lancet. 349:170.
38. Eskdale, J., G. gallagher, C. L. Vermeij, V. Keijsers, R. G. J. Westendorp and T. W. J. Huizinga. 1998. Interleukin 10 secretion in relation to human IL-10 locus haplotypes. Proc. Natl. Acad. Sci. USA. 95: 9465-9470.
39. Derkx, B., A. marchant, M. Goldman, R. Biilmer and S. Van de Venter. 1995. High levels of interleukin-10 during the initial phase of fulminant meningococcal septic shock. J. Infect. Dis. 171: 229.
40. Llorente, L., Y. Richaud-Patin, R. Fior, J. Alcocer-Varela, J. Wijdnes, B. Morel-Fourrier, P. Galanaud and P. Emilie. 1994. In vivo production of interleukin-10 by non-T cells in rheumatoid arthritis, Sjogren's syndrome, and systemic lupus erythematosus. A potential mechanism of B lymphocytes hyperactivity and autoimmunity. Arthritis Rheum. 37: 1647.
41. Cash, J. J., J. B. Splawski, R. Thomas, J. F. McFarlin, H. Schulze-Koops, L. S. Davis, K. Fujita and P. E. Lipsky. 1995. Elevated interleukin-10 levels in patients with rheumatoid arthritis. Arthritis Rheum. 38: 96.
42. Eskdale, J. P. Wordsworth, S. Bowman, M. Field and G. Gallagher. 1997. Association between polymorphisms at the human IL-10 locus and systemic lupus erythematosus. Tissue Antigens. 49: 635.
43. Zheng, C., D. Huang, L. Liu, R. Wu, S. Bergenbrant Glas, A. Ostenborg, M. Bjorkholm, G. Holm, Q. Yi, A. Sundblad. 2001. Interleukin-10 gene promoter polymorphisms in multiple myeloma. Int. J. Cancer. 95: 184.
44. Via, C. S., G. C. Tsokos, N. I. Stocks, M. Clerici and G. M. Shearer. 1990. Human in vitro allogeneic responses: demonstration of three pathways of T helper cell activation. J. Immunol. 144:2524.
45. Hahn, A. B., J. C. kasten-Jolly, D. M. Costantino, E. Graffunder, T. P. Singh, G. K. Shen and D. J. Conti. 2001. Tnf-a, Il-6, IFN-g, and IL-10 gene expression polymorphisms and the IL-4 receptor a-chain variant Q576R: effects on renal allograft outcome. Transplantation. 72: 660.
46. Lio, D., G. Candore, A. Colombo, G. Colonna Romano, F. Gervasi, V. Marino, L. Scola and C. Caruso. 2001. A genetically determined high setting of TNF-alpha influences immunologic parameters of HLA-B8, DR3 positive subjects: implications for autoimmunity. Hum Immunol. 62: 705.
47. Akdis, C. A., and K. Blaser K. 2001. Mechanisms of interleukin-10-mediated immune suppression. Immunol. 103:131.
48. Segal, B. M., B. K. Dwyer and E. M. Shevach. 1998. An interleukin (IL)-10/IL-12 immunoregulatory circuit controls susceptibility to autoimmune disease J Exp Med 187: 537.
49. Bagli M, Papassotiropuolos A, Knapp M, Jessen F. Rao M L, Maier W, Heun R. association between an interleukin-6 and 3′ flanking region haplotype and reduced Alzheimer's risk in German population. Neurosci Lett 2000; 283: 109-112.
50. Bowcock A M, Kidd J R, Lathrop G M, Daneshvar L, May L T, ray A, Sehgal P B, Kidd K K, Cvalli-Sforza L L. The human “interferon-beta 2/hepatocyte stimulating factor/IL-6” gene: DNA polymorphism studies and localization to chromosome 7p21. Genomics 1988; 3(1): 8-16.
51. Campbell I L, Stalder A K, Chiang C S, Bellinger R, Heyser C J, Steffensen S, Masliah E, Powell H C, Gold L H, henriksen S J, Siggins G R. Transgenic models to assess the pathogenic actions of cytokines in the central nervous system. Mol Psychiatry 1997; 2: 125-129.
52. Chao C C, Molitor T W, Hu S. Neuroprotective role of IL-4 against activated microglia. J Immunol 1993; 151: 1473-1481.
53. Falcone E, Spadafora P, De Luca M, Ruffolo R, Brancati C, De Benedictis G. DYS19, D12S67, and D1S80 polymorphisms in population sample from southern Italy and Greece. Hum Biol 1995; 67(5): 689-701
54. Fishman D, Faulds G, Jeffery R, Mohamed-Ali V, Yudkin J S, Humphries S, Woo P. The effect of novel polymorphisms in the interleukin-6 (IL-6) gene on IL-6 transcription and plasma IL-6 levels, and an association with systemic-onset juvenile chronic arthritis. J Clin Invest 1998; 102 (7): 1369-76.
55. Goris A, Epplen C, Fiten P, Andersson M, Murru R, sciacca F L, Ronsse I; jackel S, Epplen J T, Marrosu M G, Olsson T, Grimaldi L M, Opdenakker G. Billiau A, Vandenbroeck K. Analysis of IFN-gamma gene (IFNG) polymorphism in multiple sclerosis in Europe: effect of population structure on association with disease. J Interferon Cytokine Res 1999; 19(9): 1037-1046.
56. Kilpinen S, Hulkkonen J, Wang X Y, Hurme M. The promoter polymorphism of the IL-6 gene regulates IL-6 production in neonates but not in adults. Eur Cytokine Netw 2001; 12 (1): 62-8.
57. Kim J M, Brannan C I, Copeland N G, Jenkins N A, Khan T A, Moore K W. Structure of the mouse IL-10 gene and chromosomal localisation of the mouse and human genes. Journal of Immunology 1992; 148: 3618.
58. Lio D, Scola L, Crivello A, Colonna-Romano G, Candore G, Bonafè M, Cavallone L, Franceschi C, Caruso C. Gender-specific association between −1082 IL-10 promoter polymorphism and longevity. Genes and Immunity 2002; 3: 30-33.
59. Llorente L, Richaud-Patin Y, Couderc J, Alarcon-Segovia D, Ruiz-Soto R, Alcocer-Castillejos N, Alcocer-Varela J, Granados J, Bahena S, Galanaud P, Emilia D. Dysregulation of interleukin-10 production in relatives of patients with systemic lupus erythematosus. Arthritis Rheum 1997;38(8): 1429-35.
60. Mateo I, Sanchez-Guerra M, Combarros O, Llorca J, Infante J, Gonzalez-Garcia J. del Molino J P, Berciano J. Lack of association between cathepsin D genetic polymorphism and Alzheimer disease in a Spanish sample. Am J Med Genet 2002; 114 (1): 31-3.
61. Murray R E, McGuigan F, Grant S F, Reid D M, Ralston S H. Polymorphisms of the interleukin-6 gene are associated with bone mineral density. Bone 1997; 21: 89-92.
62. Pallaud C, Stranieri C, Sass C, Siest G, Pignatti F, Visvikis S. Candidate gene polymorphimss in cardiovascular disease: a comparative study of frequencies between a French and an Italian population. Clin Chem Lab Med 2001; 39(2): 146-54.
63. Papassotiropoulos A, Hock C, Nitsch R M. Genetics of interleukin 6: implications for Alzheimer's disease. Neurobiology of Aging 1999;22: 863-871.
64. Pola R, Flex A, Gaetani E, Dal Lago A, Gerardini L, Pola P, Bernabei R. The −174 G/C polymorphism of the interleukin-6 gene promoter is associated with Alzheimer's disease in an Italian population. Neuroreport 2002; 13: 1645-1647.
65. Poli F, Nocco A, Berra S, Scalamogna M, Taioli E, Longhi, Sirchia G. Allele frequencies of polymorphisms of TNF-α, Il-6, Il-10 and IFNG in an Italian Caucasian population. European journal of Immunogenetics 2002; 29: 237-240.
66. Shibata N, Ohnuma T, Takahashi T, Baba H, Ishizuka T, Ohtsuka M, Ueki A, Nagao M, Arai H. Effect of IL-6 polymorphism on risk of Alzheimer disease: genotype-phenotype association study in Japanese cases. American Journal of Medical Genetics 2002; 114: 436-439.
67. Strauss S, Bauer J, Ganter U, Jonas U, Berger M, Volk B. detection of IL-6 and alpha 2-macroglobulin immunoreactivity in cortex and hippocampus of Alzheimer's disease patients. Lab Invest 1992; 66: 223-30.
68. Terry C F, Loukaci V, Green F R. Cooperative influence of genetic polymorphisms on interleukin 6 transcriptional regulation. J Biol Chem 2000; 275 (24): 18138-44.
69. Vandenabeele P, Fiers W. Is amyloidogenesis during Alzheimer's disease due to an IL-I/IL-6 mediated “acute phase response” in the brain? Immunol Today 1991;12:217-19.
70. Vandenbroeck K, Hardt C, Louage j, Fiten P, Jackel S, Ronsse I, Epplen J T, Grimaldi L M, Olsson T, Marrosu M G, Billiau A, Opdenakker G. Lack of association between the interferon regulatory factor-1 (IRF1) locus at 5p31.1 and multiple sclerosis in Germany, northern Italy, Sardinia and Sweden. Genes Immun 2000; 1(4): 290-2.
71. Quintana F J. Carmi P. Cohen I R. DNA vaccination with heat shock protein 60 inhibits cyclophosphamide-accelerated diabetes. Journal of Immunology. 169(10):6030-5, 2002 Nov. 15.
72. Dalpke A H. Opper S. Zimmermann S. Heeg K. Suppressors of cytokine signaling (SOCS)-1 and SOCS-3 are induced by CpG-DNA and modulate cytokine responses in APCs. Journal of Immunology. 166(12):7082-9, 2001 Jun. 15.
73. Yi A K. Yoon J G. Yeo S J. Hong S C. English B K. Krieg A M. Role of mitogen-activated protein kinases in CpG DNA-mediated IL-10 and IL-12 production: central role of extracellular signal-regulated kinase in the negative feedback loop of the CpG DNA-mediated Th1 response. Journal of Immunology. 168(9):4711-20, 2002 May 1.
74. G. Hartmann et al., “CpG DNA: A potent signal for growth, activation, and maturation of human dendritic cells,” Proceedings of the National Academy of Sciences, 96:9305-10, 19975. Hollon T. Coley Toxin's Hidden message: from a 19^{th} century mystery comes a potential new class of drugs: CpG oligonucleotides. The Scientist 15(5): 2001, Mar. 5.
76. Hybridon's press release on May 07, 2003 on data presented during the First Annual Meeting of the Federation of Clinical Immunology Societies held in Boston on May 4 to May 7, 2001, Posters#271 “Modulation of immunostimulatory activity of CpG-oligonucleotides by incorporation of site-specific chemical modifications: Significance of internucleoside negative charge” Poster #229 “Oligonucleotides containing YpG or CpR motifs as potent immunostimulatory agents”, found on: http://www.nrp-euro.com.
77. Du Y. et al. Association of an interleukin 1 alpha polymorphism with Alzheimer's disease. Neurology 55(4): 464-5 2000, Aug. 22

1. A method of determining the existence of or a predisposition to Alzheimer's disease, autoimmune disease or other neurodegenerative diseases, the method comprising analysing a DNA bearing sample taken from a subject animal to determine the allelic variants present at one or more of the SNP loci at positions −1082, −819 and −592 of the gene encoding IL-10.

2. A method according to claim 1, in which the genotype at all three positions −1082, −819 and −592 is determined.

3. A method according to claim 1 which further comprises analysing the sample to determine the alleles present for the genes encoding IL-6 and Apo-E.

4. A method according to claim 3 which further comprises analysing the sample to determine the alleles present for the gene encoding IL-1.

5. A method of treating Alzheimer's disease, autoimmune disease or other neurodegenerative disorder which comprises augmenting the function of a gene having one of the allelic polymorphisms of IL-10 shown in Table I.

6. A method of treating Alzheimer's disease, autoimmune disease or other neurodegenerative disorder which comprises decreasing the function of a gene having one of the allelic polymorphisms of IL-10 shown in Table I.

7. A method according to claim 5 where the modulation of the function of the gene is by genetic therapy.

8. A method according to claim 5 where the modulation of the function of the gene is by pharmacological intervention.

9. A method according to claim 8 where the pharmacological intervention is using one or more compounds that enhance or inhibit antigen specific production of interleukin-10 and, optionally, one or more other cytokines.

10. A method according to claim 9, characterised in that the other cytokine is selected from the group consisting of interleukin-1 (α or β), interleukin-2, interleukin-3, interleukin-4, interleukin-5, interleukin-6, interleukin-7, interleukin-8, interleukin-9, interleukin-11, interleukin-12, interleukin-13, interleukin-14, interleukin-15, interleukin-16, interleukin-17, interferon-α, interferon-β, interferon-γ, TNF-α, TNF-β, G-CSF, GM-CSF, M-LSF, and TGF-β.

11. DNA fragments and cDNA fragments comprising the allelic polymorphs of Table I for use in the method of claim 7.

12. Use of the DNA or cDNA fragments of claim 11 in a method of screening compounds for the ability to modulate the allelic polymorphisms of Table I.

13. Use of the DNA or cDNA fragments of claim 11 in a method of screening compounds for the ability to modulate or prevent Alzheimer's disease.

14. Use of cytokines in the preparation of a medicament for the treatment or prophylaxis of diseases which are not neoplastic.

15. Use according to claim 14, characterised in that the disease is a neurodegenerative disorder or an autoimmune disorder.

16. Use according to claim 14, characterised in that the use is for Alzheimer's disease.

17. Use according to any one of claim 14, characterised in that the cytokine is selected from interleukin-1 (α or β), interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-15, interleukin-16, interleukin-17, interferon-α, interferon-β, interferon-γ, TNF-α, TNF-β, G-CSF, GM-CSF, M-LSF, and TGF-β.

18. A method according to claim 6 where the modulation of the function of the gene is by genetic therapy.

19. A method according to claim 6 where the modulation of the function of the gene is by pharmacological intervention.

20. A method according to claim 19 where the pharmacological intervention is using one or more compounds that enhance or inhibit antigen specific production of interleukin-10 and, optionally, one or more other cytokines.
